(19) United States
(12) Patent Application Publication
(10) Pub. No.: US 2026/0301906 A1
(43) Pub. Date: Oct. 1, 2026

(54) MEDICATION STORAGE LOCKER SYSTEM AND METHOD THEREFOR

(71) Applicant: Lindsey Clark, Findlay, OH (US)

(72) Inventor: Lindsey Clark, Findlay, OH (US)

(21) Appl. No.: 19/097,034

(22) Filed: Apr. 1, 2025

(51) Int. Cl. A47B 67/00; G16H 20/13

(52) U.S. Cl. CPC G16H 20/13; A47B 67/00

(57) ABSTRACT

A medication storage locker system for customers of a pharmacy includes a housing, a plurality of doors, a plurality of electronic locks, a server, a computer-program product, an electronic interface, a microprocessor, and a power source. The housing includes a plurality of compartments with doors having electronic locks. The server stores information relating to a medication, a customer, a designated compartment in which the medication is stored, and a random access code to open the designated compartment. The computer-program product is on a personal electronic device and communicates with the server to receive and display the random access code, which can be input via the electronic interface on the housing. The microprocessor is connected to the electronic locks. The microprocessor is in communication with the server to receive the random access code to disengage the electronic lock of the designated compartment.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to secure storage lockers for medication and more particularly pertains to a new medication storage locker system for securely storing medication for retrieval by a customer of a pharmacy. Usually, when customers pick up medication at a pharmacy, the customer must wait in a line to be personally given the medication by a member of the pharmacy staff, often a pharmacist who provides information about the medication and verifies whether or not the customer has any questions about the medication. The new medication storage locker system allows pharmacy staff to store the medication in locked compartments that are openable only upon entry of a code by the customer, thereby allowing the customer to retrieve the medication in a secure manner and skip having to wait in line.

### (2) Description of Related Art including information disclosed under 37 CFR 1.97 and 1.98.

The prior art relates to secure storage lockers for medication. The prior art, as best understood, does not disclose lockboxes in one location, a code for opening the lockbox, and a mobile application used for communication, approval, and payment.

### (h) BRIEF SUMMARY OF THE INVENTION

An embodiment of the disclosure meets the needs presented above in a medication storage locker system for customers of a pharmacy generally comprising a housing, a plurality of doors, a plurality of electronic locks, a server, a computer-program product, an electronic interface, a microprocessor, and a power source. The housing includes a front wall, a rear wall, and a perimeter wall. The housing includes a plurality of compartments open at the front wall. Each of the doors is movably coupled to the front wall of the housing to cover a respective one of the compartments. Each of the electronic locks is designed to lock a respective one of the doors. The server is designed to receive and store information relating to a medication, a customer, a designated compartment of the compartments in which the medication is stored, and a random access code to open the designated compartment. The computer-program product is tangibly embodied in a non-transitory machine-readable storage medium of a personal electronic device. The computer-program product is designed to communicate with the server to receive and display the random access code. The electronic interface is mounted on the housing. The electronic interface is designed for input of the random access code. The microprocessor is disposed in the housing. The microprocessor is connected to the electronic locks. The microprocessor is in communication with the server to receive the random access code to disengage the electronic lock of the designated compartment. The power source is designed to supply power to the microprocessor and the electronic locks.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a medication storage locker system according to an embodiment of the disclosure.

FIG. 2 is a front view of an embodiment of the disclosure.

FIG. 3 is a rear view of an embodiment of the disclosure.

FIG. 4 is a cross-sectional view of a component of the embodiment of the disclosure in FIG. 3.

FIG. 5 is a cross-sectional view of a component of the embodiment of the disclosure in FIG. 4 in use.

FIG. 6 is a cross-sectional view of a component of an alternative embodiment of the disclosure.

FIG. 7 is a block diagram of an embodiment of the disclosure.

FIG. 8 is a flow chart showing steps of operation of an embodiment of the disclosure.

### (j) DETAILED DESCRIPTION OF THE INVENTION

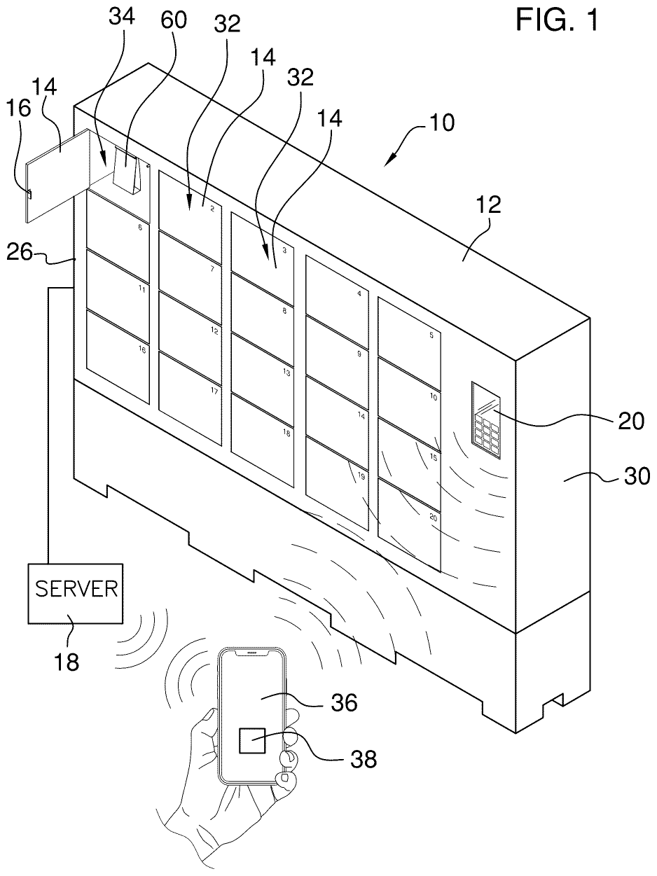

With reference now to the drawings, and in particular to FIGS. 1 through 8 thereof, a new medication storage locker system embodying the principles and concepts of an embodiment of the disclosure and generally designated by the reference numeral 10 will be described.

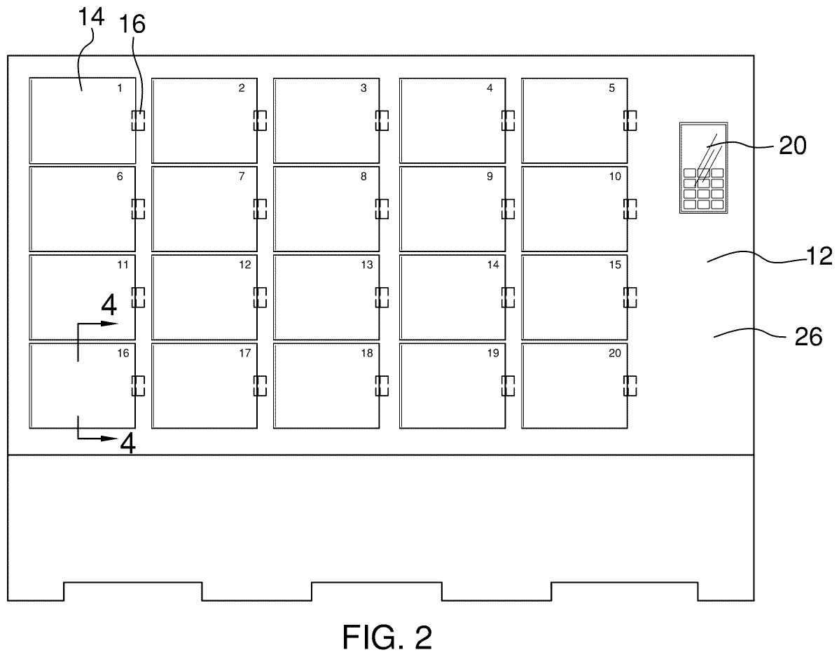

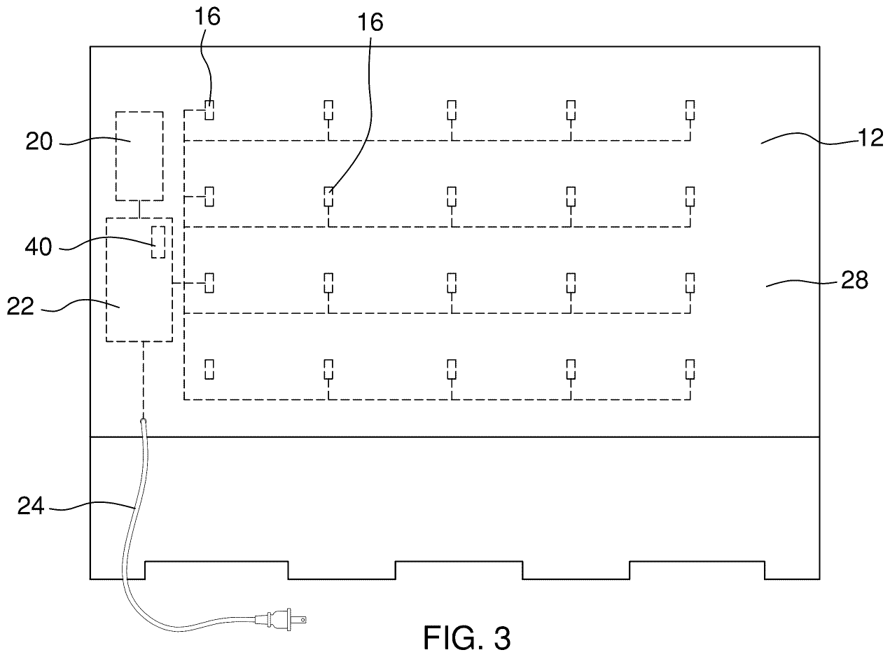

As best illustrated in FIGS. 1 through 8, the medication storage locker system 10 generally comprises a housing 12, a plurality of doors 14, a plurality of electronic locks 16, a server 18, a computer-program product, an electronic interface 20, a microprocessor 22, and a power source 24. The housing 12 includes a front wall 26, a rear wall 28, and a perimeter wall 30. The housing 12 includes a plurality of compartments 32 open at the front wall 26. Each of the doors 14 is movably coupled to the front wall 26 of the housing 12 to cover a respective one of the compartments 32. As shown in FIG. 2, each of the electronic locks 16 is designed to lock a respective one of the doors 14. The server 18 is designed to receive and store information relating to a medication 60, a customer, a designated compartment 34 of the compartments 32 in which the medication 60 is stored, and a random access code to open the designated compartment 34. The computer-program product is tangibly embodied in a non-transitory machine-readable storage medium 38 of a personal electronic device 36. The computer-program product is designed to communicate with the server 18 to receive and display the random access code. The electronic interface 20 is mounted on the housing 12. The electronic interface 20 is designed for input of the random access code. As shown in FIG. 3, the microprocessor 22 is disposed in the housing 12. The microprocessor 22 is connected to the electronic locks 16. The microprocessor 22 is in communication with the server 18 to receive the random access code to disengage the electronic lock of the designated compartment 34. The power source 24 is designed to supply power to the microprocessor 22 and the electronic locks 16.

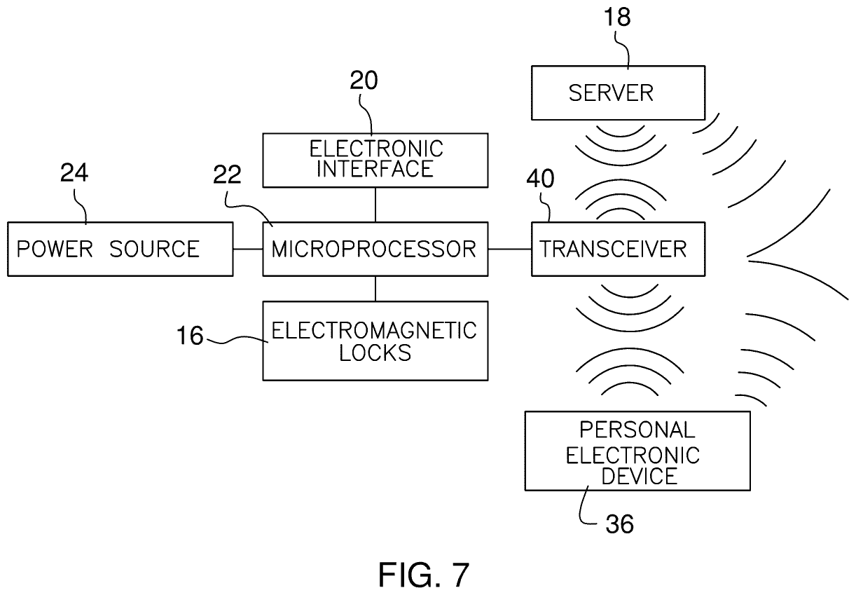

In the exemplary embodiment in FIG. 3, the medication storage locker system 10 further includes a transceiver 40. The transceiver 40 is positioned in the housing 12. The transceiver 40 is connected to the microprocessor 22 for wireless communication with the server 18 and the personal electronic device 36, as shown in FIG. 7. In another possible embodiment, the microprocessor 22 could be connected to the server 18 by a hardwired connection, as shown in FIG. 1. The connection between the microprocessor 22 and the server 18 is for transmission of the information mentioned above. In another possible embodiment, the connection between the microprocessor 22 and the personal electronic device 36 could be for transmission of this information. In other words, the server 18 transmits the information to the computer-program product on the personal electronic device 36, such as a smartphone as shown in FIG. 1, and the personal electronic device 36 then transmits the information to the microprocessor 22. In another possible embodiment, the computer-program product wirelessly transmits location information to the microprocessor 22 to verify that the customer is within a certain distance of the microprocessor 22, which verification can function as another layer of security to ensure only the customer receives the medication 60.

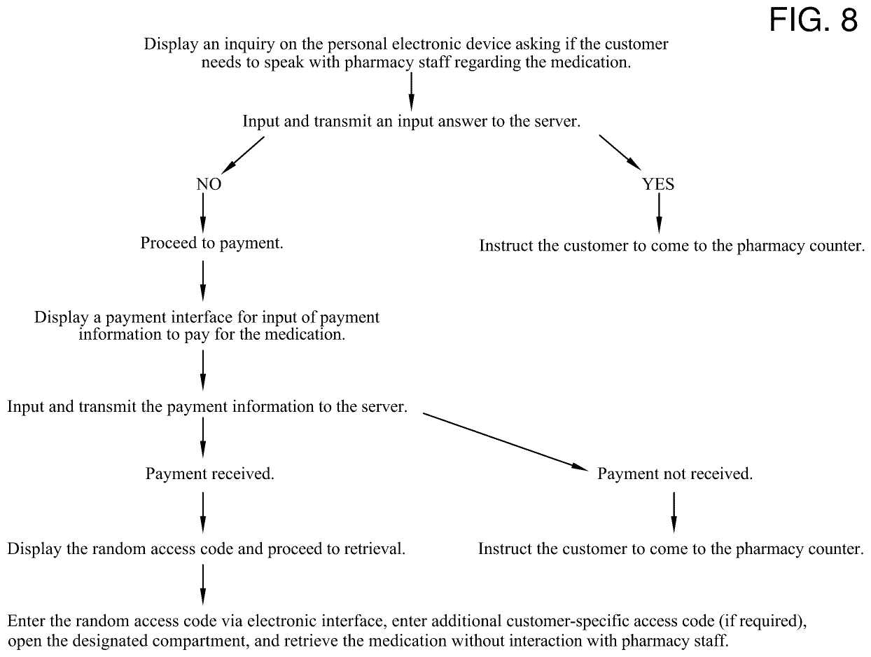

As shown in FIG. 8, the computer-program product is designed to display an inquiry asking if the customer needs to speak with pharmacy staff regarding the medication 60. The computer-program product is designed to transmit an input answer to the server 18. The computer-program product is designed to display the random access code upon the input answer indicating the customer does not need to speak with pharmacy staff.

Also as shown in FIG. 8, the computer-program product is designed to display a payment interface for input of payment information to pay for the medication 60. The computer-program product is designed to transmit the payment information to the server 18. The computer-program product is designed to display the random access code upon the transmission of the payment information to the server 18.

In the exemplary embodiment in FIG. 8, the microprocessor 22 is designed to open the electronic locks 16 only upon input of a customer-specific access code stored on the server 18 in addition to the random access code, wherein the customer-specific access code is one of: the customer's birthdate, the customer's driver's license number, and the customer's social security number. However, this is one possible embodiment, and this additional verification could be omitted and only the random access code used.

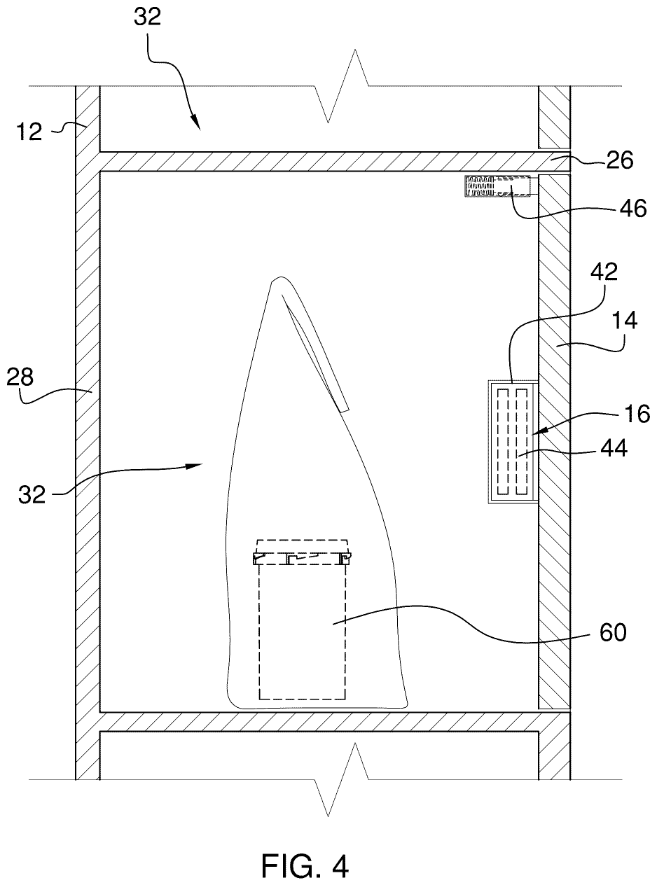

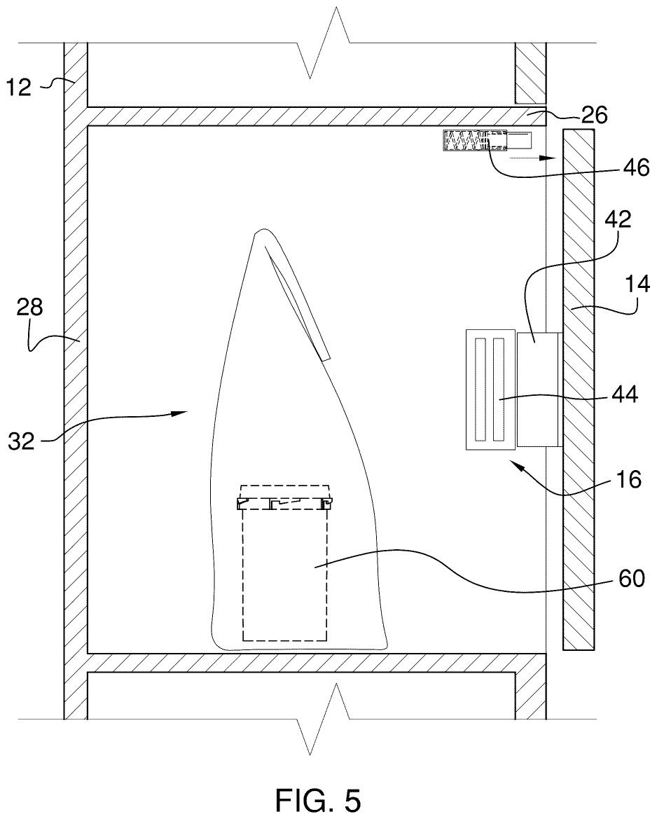

Each of the electronic locks 16 includes a metal strike plate 42 and an electromagnet 44 designed to magnetically engage the metal strike plate 42 to hold the door in place by magnetic force. In the exemplary embodiment in FIGS. 4 and 5, the medication storage locker system 10 further includes a plurality of biasing springs 46. Each of the biasing springs 46 is designed to push open a respective one of the doors 14 upon disengagement of a respective one of the electronic locks 16. It should be noted that this is only one exemplary embodiment, and other designs are possible, such as doors 14 with turnable lock handles or automated sliding doors 14.

In the exemplary embodiment in FIG. 1, the electronic interface 20 is a physical keypad or a digital screen displaying a virtual keypad.

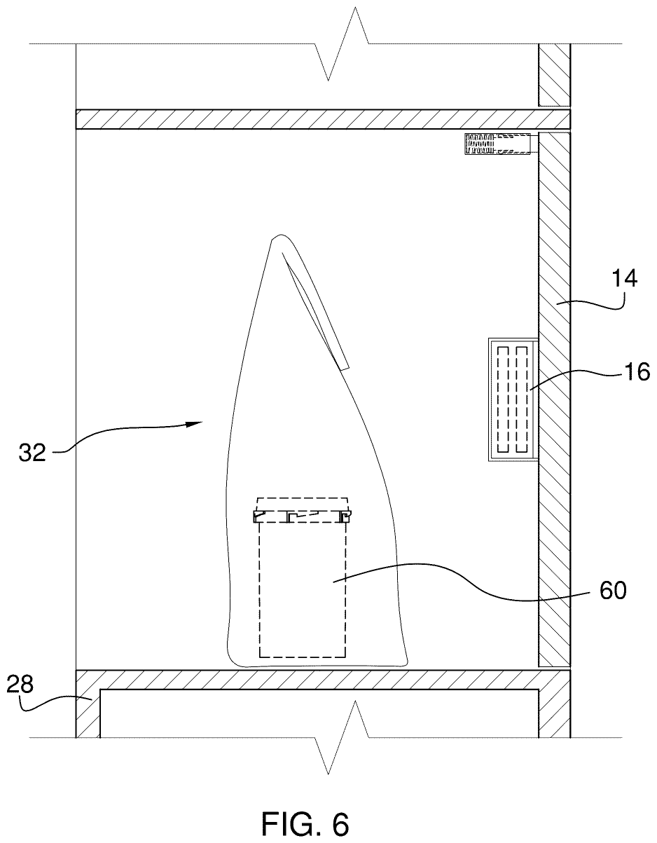

In the exemplary embodiment in FIG. 6, each of the compartments 32 is additionally open in the rear wall 28 of the housing 12 for insertion of medication 60 into the compartments 32. To further explain, in the exemplary embodiment in FIGS. 1 through 5, the compartments 32 are closed on all sides except for on the front wall 26 where the doors 14 are located. Accordingly, pharmacy staff must place the medication 60 into the compartments 32 via the doors 14. In such a situation, the doors 14 could be always open until closed with medication 60 inside, or possibly openable with a master code or master key. In the exemplary alternative embodiment in FIG. 6, the compartments 32 are open in the rear wall 28 to allow pharmacy staff to insert the medication 60 from behind. In such an embodiment, the housing 12 could be integrated into a wall of the pharmacy dispensary, and the pharmacy staff working in the dispensary could simply and securely place the medication 60 into the locked compartments 32. After the customer retrieves the medication 60, the customer closes the door 14, thereby returning the door 14 to a locked state.

The medication storage locker system 10 is used to securely store the medication 60 for the customers of the pharmacy. In the exemplary embodiment, information relating to the medication 60, the customer, and a designated compartment 34 of the compartments 32 in which the medication 60 is stored is entered and stored on the server 18. The random access code to open the designated compartment 34 is generated and communicated to the microprocessor 22. The pharmacy staff then places the medication 60 in the designated compartment 34. The customer uses the computer-program product on the personal electronic device 36 to communicate with the server 18 to receive and display the random access code. The customer then inputs the random access code into the electronic interface 20 and thereby disengages the electronic lock of the designated compartment 34. The door is now openable or pops open by force of the biasing spring. The customer may now retrieve the medication 60 from the designated compartment 34 without interaction between the customer and pharmacy staff.

In the exemplary embodiment, the computer-program product displays an inquiry on the personal electronic device 36 asking if the customer needs to speak with pharmacy staff regarding the medication 60, as is often the case in pharmacy operations. The customer inputs an answer that is then transmitted to the server 18. The random access code is displayed only if the input answer indicates the customer does not need to speak with pharmacy staff. If the customer does need to speak with the pharmacy staff, the server 18 will not supply the random access code and the customer will be required to visit the pharmacy counter to receive the medication 60.

In the exemplary embodiment, the computer-program product also displays a payment interface for input of payment information to pay for the medication 60. The customer inputs the payment information that is transmitted to the server 18, or possibly selects a payment option stored on the server 18. If the customer transmits the payment information and thus successfully pays for the medication 60, the computer-program product will display the random access code. If the customer does not successfully pay for the medication 60, the server 18 will not supply the random access code and the customer will be required to visit the pharmacy counter to receive the medication 60.

In one possible embodiment described herein, the microprocessor 22 can be designed to open the electronic locks 16 only upon input of a customer-specific access code stored on the server 18 in addition to the random access code, wherein the customer-specific access code is one of: the customer's birthdate, the customer's driver's license number, and the customer's social security number. In this particular embodiment, the customer would additionally have to input the customer-specific access code into the electronic interface 20.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A medication storage locker system for customers of a pharmacy comprising:
a housing, the housing including a front wall, a rear wall, and a perimeter wall, the housing including a plurality of compartments open at the front wall;
a plurality of doors, each of the doors being movably coupled to the front wall of the housing to cover a respective one of the compartments;
a plurality of electronic locks, each of the electronic locks being configured to lock a respective one of the doors;
a server, the server being configured to receive and store information relating to a medication, a customer, a designated compartment of the compartments in which the medication is stored, and a random access code to open the designated compartment;
a computer-program product, the computer-program product being tangibly embodied in a non-transitory machine-readable storage medium of a personal electronic device, the computer-program product being configured to communicate with the server to receive and display the random access code;
an electronic interface, the electronic interface being mounted on the housing, the electronic interface being configured for input of the random access code;
a microprocessor, the microprocessor being disposed in the housing, the microprocessor being connected to the electronic locks, the microprocessor being in communication with the server to receive the random access code to disengage the electronic lock of the designated compartment; and
a power source, the power source being configured to supply power to the microprocessor and the electronic locks.

2. The medication storage locker system of claim 1, further comprising a transceiver, the transceiver being positioned in the housing, the transceiver being connected to the microprocessor for wireless communication with the server and the personal electronic device.

3. The medication storage locker system of claim 1, wherein:
the computer-program product is configured to display an inquiry asking if the customer needs to speak with pharmacy staff regarding the medication;
the computer-program product is configured to transmit an input answer to the server; and
the computer-program product is configured to display the random access code upon the input answer indicating the customer does not need to speak with pharmacy staff.

4. The medication storage locker system of claim 1, wherein:
the computer-program product is configured to display a payment interface for input of payment information to pay for the medication;
the computer-program product is configured to transmit the payment information to the server; and
the computer-program product is configured to display the random access code upon the transmission of the payment information to the server.

5. The medication storage locker system of claim 1, wherein the microprocessor is configured to open the electronic locks only upon input of a customer-specific access code stored on the server in addition to the random access code, wherein the customer-specific access code is one of: the customer's birthdate, the customer's driver's license number, and the customer's social security number.

6. The medication storage locker system of claim 1, wherein each of the electronic locks includes a metal strike plate and an electromagnet configured to magnetically engage the metal strike plate to hold the door in place by magnetic force.

7. The medication storage locker system of claim 1, further comprising a plurality of biasing springs, each of the biasing springs being configured to push open a respective one of the doors upon disengagement of a respective one of the electronic locks.

8. The medication storage locker system of claim 1, wherein the electronic interface is a physical keypad or a digital screen displaying a virtual keypad.

9. The medication storage locker system of claim 1, wherein each of the compartments is additionally open in the rear wall of the housing for insertion of medication into the compartments.

10. The medication storage locker system of claim 2, wherein:
the computer-program product is configured to display an inquiry asking if the customer needs to speak with pharmacy staff regarding the medication;
the computer-program product is configured to transmit an input answer to the server; and
the computer-program product is configured to display the random access code upon the input answer indicating the customer does not need to speak with pharmacy staff.

11. The medication storage locker system of claim 10, wherein:
the computer-program product is configured to display a payment interface for input of payment information to pay for the medication;
the computer-program product is configured to transmit the payment information to the server; and
the computer-program product is configured to display the random access code upon the transmission of the payment information to the server.

12. The medication storage locker system of claim 11, wherein the microprocessor is configured to open the electronic locks only upon input of a customer-specific access code stored on the server in addition to the random access code, wherein the customer-specific access code is one of: the customer's birthdate, the customer's driver's license number, and the customer's social security number.

13. The medication storage locker system of claim 12, wherein each of the electronic locks includes a metal strike plate and an electromagnet configured to magnetically engage the metal strike plate to hold the door in place by magnetic force.

14. The medication storage locker system of claim 13, further comprising a plurality of biasing springs, each of the biasing springs being configured to push open a respective one of the doors upon disengagement of a respective one of the electronic locks.

15. The medication storage locker system of claim 14, wherein the electronic interface is a physical keypad or a digital screen displaying a virtual keypad.

16. A method of securely storing medication for customers of a pharmacy, the method comprising the steps of:
providing a medication storage locker system for customers of a pharmacy comprising:
a housing, the housing including a front wall, a rear wall, and a perimeter wall, the housing including a plurality of compartments open at the front wall;
a plurality of doors, each of the doors being movably coupled to the front wall of the housing to cover a respective one of the compartments;
a plurality of electronic locks, each of the electronic locks being configured to lock a respective one of the doors;
a server, the server being configured to receive and store information relating to a medication, a customer, a designated compartment of the compartments in which the medication is stored, and a random access code to open the designated compartment;
a computer-program product, the computer-program product being tangibly embodied in a non-transitory machine-readable storage medium of a personal electronic device, the computer-program product being configured to communicate with the server to receive and display the random access code;
an electronic interface, the electronic interface being mounted on the housing, the electronic interface being configured for input of the random access code;
a microprocessor, the microprocessor being disposed in the housing, the microprocessor being connected to the electronic locks, the microprocessor being in communication with the server to receive the random access code to disengage the electronic lock of the designated compartment; and
a power source, the power source being configured to supply power to the microprocessor and the electronic locks;
entering and storing information relating to a medication, a customer, and a designated compartment of the compartments in which the medication is stored;
generating a random access code to open the designated compartment and communicating the random access code to the microprocessor;
placing the medication in the designated compartment;
communicating with the server to receive and display the random access code via the computer-program product on the personal electronic device;
inputting the random access code into the electronic interface and thereby disengaging the electronic lock of the designated compartment; and
retrieving the medication from the designated compartment without interaction between the customer and pharmacy staff.

17. The method of claim 16, further comprising the steps of:
displaying an inquiry on the personal electronic device asking if the customer needs to speak with pharmacy staff regarding the medication;
inputting and transmitting an input answer to the server; and
displaying the random access code upon the input answer indicating the customer does not need to speak with pharmacy staff.

18. The method of claim 17, further comprising the steps of:
displaying a payment interface for input of payment information to pay for the medication;
inputting and transmitting the payment information to the server; and
displaying the random access code upon the transmission of the payment information to the server.

19. The method of claim 18, wherein:
the microprocessor is configured to open the electronic locks only upon input of a customer-specific access code stored on the server in addition to the random access code, wherein the customer-specific access code is one of:
the customer's birthdate, the customer's driver's license number, and the customer's social security number; and
said step of inputting the random access code into the electronic interface further includes additionally inputting the customer-specific access code into the electronic interface.
